(19) United States
(12) Patent Application Publication
(10) Pub. No.: US 2008/0089953 A1
(43) Pub. Date: Apr. 17, 2008

(54) Self-Preserved Ophthalmic Pharmaceutical Compositions Containing Tobramycin

(75) Inventors: Masood A. Chowhan, Arlington, TX (US); Ernesto J. Castillo, Fort Worth, TX (US); Glenn D. Stafford Jr., White Settlement, TX (US); Rajni Jani, Fort Worth, TX (US); Haresh G. Bhagat, Fort Worth, TX (US)

Correspondence Address: ALCON, IP LEGAL, TB4-8, 6201 SOUTH FREEWAY, FORT WORTH, TX, 76134, US

(73) Assignee: ALCON, INC., Hunenberg (CH)

(21) Appl. No.: 11/955,046

(22) Filed: Dec. 12, 2007

Related U.S. Application Data
(63) Continuation of application No. 11/317,261, filed on Dec. 23, 2005, now abandoned.
(60) Provisional application No. 60/639,421, filed on Dec. 27, 2004.

(51) Int. Cl. A61K 31/7008; A61K 33/22; A61P 31/04

(52) U.S. Cl. 424/660; 514/38

(57) ABSTRACT

Self-preserved, multi-dose ophthalmic compositions containing tobramycin are described. The compositions do not contain a conventional antimicrobial preservative, such as benzalkonium chloride. Rather, the compositions are self-preserved as a result of the inherent antimicrobial activity of tobramycin. The compositions preferably also contain either boric acid or, more preferably, a borate/polyol complex selected from the group consisting of borate/propylene glycol, borate/glycerol, and combinations thereof.

### CLAIM FOR PRIORITY

The present application is a continuation of U.S. patent application Ser. No. 11/317,261 filed Dec. 23, 2005, which claims priority based on U.S. Provisional Patent Application Ser. No. 60/639,421, filed Dec. 27, 2004.

### BACKGROUND OF THE INVENTION

The present invention is directed to multi-dose, aqueous ophthalmic pharmaceutical compositions that are self-preserved as a result of the inherent antimicrobial activity of the antibiotic tobramycin. The anti-microbial activity and preservative efficacy of the compositions is enhanced by certain preferred formulation criteria described herein.

Many pharmaceutical compositions are required to be sterile (i.e., free of bacteria, fungi and other pathogenic microorganisms). Examples of such compositions include: solutions and suspensions that are applied into the bodies of humans or other mammals; creams, lotions, solutions or other preparations that are topically applied to wounds, abrasions, burns, rashes, surgical incisions, or other conditions where the skin is not intact; and various types of compositions that are applied either directly to the eye (e.g., artificial tears, irrigating solutions, and drug products), or are applied to devices that will come into contact with the eye (e.g., contact lenses).

The foregoing types of compositions can be manufactured under sterile conditions via procedures that are well known to those skilled in the art. However, once the packaging for a product is opened, such that the composition contained therein is exposed to the atmosphere and other sources of potential microbial contamination (e.g., the hands of a human patient), the sterility of the product may be compromised. Such products are typically utilized multiple times by the patient, and are therefore frequently referred to as being of a “multi-dose” nature.

Due to the frequent, repeated exposure of multi-dose products to the risk of microbial contamination, it is necessary to employ a means for preventing such contamination from occurring. The means employed may be: (i) an antimicrobial agent that prevents the proliferation of microbes in a composition, which is referred to herein as an “antimicrobial preservative”; or (ii) a packaging system that prevents or reduces the risk of microbes reaching a pharmaceutical composition within a container.

Prior multi-dose ophthalmic compositions have generally contained one or more antimicrobial preservatives in order to prevent the proliferation of bacteria, fungi and other microbes. Such compositions may come into contact with the cornea either directly or indirectly. The cornea is particularly sensitive to exogenous chemical agents. Consequently, in order to minimize the potential for harmful effects on the cornea, it is preferable to use anti-microbial preservatives that are relatively non-toxic to the cornea, and to use such preservatives at the lowest possible concentrations (i.e., the minimum amounts required in order to perform their anti-microbial functions).

Balancing the anti-microbial efficacy and potential toxicological effects of anti-microbial preservatives is sometimes difficult to achieve. More specifically, the concentration of necessary for the preservation of ophthalmic formulations from microbial contamination may create the potential for toxicological effects on the cornea and/or other ophthalmic tissues. Using lower concentrations of the anti-microbial agents generally helps to reduce the potential for such toxicological effects, but the lower concentrations may be insufficient to achieve the required level of biocidal efficacy (i.e., antimicrobial preservation).

The use of an inadequate level of antimicrobial preservation may create the potential for microbial contamination of the compositions and ophthalmic infections resulting from such contaminations. This is also a serious problem, since ophthalmic infections involving Pseudomonas aeruginosa or other virulent microorganisms can lead to loss of visual function or even loss of the eye.

Ophthalmic compositions are generally formulated as isotonic, buffered solutions. One approach to enhancing the anti-microbial activity of such compositions is to include multi-functional components in the compositions. In addition to performing their primary functions, these multi-functional components also serve to enhance the overall anti-microbial activity of the compositions.

The following publications may be referred to for further background regarding the use of multi-functional components to enhance the antimicrobial activity of ophthalmic compositions:
1. U.S. Pat. No. 5,817,277 (Mowrey-McKee, et al. tromethamine);
2. U.S. Pat. No. 6,503,497 (Chowhan, et al.; borate/polyol complexes);
3. U.S. Pat. No. 5,741,817 (Chowhan, et al.; low molecular weight amino acids such as glycine);
4. U.S. Pat. No. 6,319,464 (Asgharian; low molecular weight amino alcohols); and
5. U.S. Patent Application Publication No. US 2002/0122831 A1 (Mowrey-McKee, et al.; bis-aminopolyols).

The compositions of the present invention are multi-dose products that do not contain a conventional antimicrobial preservative (e.g., benzalkonium chloride), but yet are preserved from microbial contamination. Such compositions have been referred to in the art as being “preservative free” (see, e.g., U.S. Pat. No. 5,597,559 issued to Olejnik, et al.). Compositions that are preserved from microbial contamination as a result of the inherent antimicrobial activity of one or more components of the compositions are also referred to in the art as being “self-preserved” (see, e.g., U.S. Pat. No. 6,492,361 issued to Muller, et al.).

The following publication may be referred to for further background regarding pharmaceutical compositions that are “preservative-free” or “self-preserving”: Kabara, et al., Preservative-Free and Self-Preserving Cosmetics and Drugs—Principles and Practice, Chapter 1, pages 1-14, Marcel Dekker, Inc. (1997).

The antibiotic tobramycin has been utilized in ophthalmic pharmaceutical products for many years. A 0.3% tobramycin solution is currently marketed by Alcon Laboratories, Inc. under the name “TOBREX®”. An aqueous suspension containing 0.3% tobramycin and 0.1% dexamethasone is currently marketed by Alcon Laboratories, Inc. under the name “TOBRADEX®”. The latter product is described in U.S. Pat. No. 5,149,694. Both of the above-mentioned products are preserved by means of benzalkonium chloride.

The present invention is directed to the provision of improved tobramycin compositions that do not contain benzalkonium chloride or other conventional antimicrobial preservatives.

### SUMMARY OF THE INVENTION

The present invention is based on the discovery that an aqueous ophthalmic composition containing tobramycin at a concentration of 0.3 percent by weight/volume has sufficient antimicrobial activity to satisfy the United States Pharmacopoeia (“USP”) standards for preservation of aqueous, multi-dose ophthalmic compositions, particularly the USP 27 Antimicrobial Effectiveness Test, without the use of a conventional anti-microbial preservative, such as benzalkonium chloride (“BAC”). The multi-dose compositions of the present invention do not contain such conventional preservatives, and therefore are referred to herein as being “self-preserved”.

The tobramycin compositions of the present invention preferably contain boric acid and/or a pharmaceutically acceptable salt thereof (e.g., sodium borate) to enhance the anti-fungal activity of the compositions, and most preferably contain a selected type of borate/polyol complex to further enhance the antimicrobial activity of the compositions.

With respect to the preferred embodiment of the invention, wherein a borate/polyol complex is utilized, the invention was achieved as a result of a balancing of the need to provide sufficient antimicrobial activity to satisfy the USP 27 Antimicrobial Effectiveness Test, while maintaining the chemical stability of tobramycin. More specifically, initial attempts to satisfy the above-cited USP preservative efficacy requirements by means of the inclusion of a borate/polyol complex (i.e., boric acid/sorbitol) in a tobramycin composition were successful, relative to microbiological criteria, but resulted in an adverse effect on the chemical stability of tobramycin. The present invention is based on the surprising finding that other types of borate/polyol complexes (i.e., borate/propylene glycol and borate/glycerin) are capable of enhancing the antimicrobial activity of aqueous tobramycin compositions without compromising the chemical stability of tobramycin. The self-preserved tobramycin compositions of the present invention therefore contain these selected types of borate/polyol complexes.

The present invention is particularly directed to the provision of self-preserved ophthalmic compositions containing a combination of tobramycin and an anti-inflammatory agent (e.g., a corticosteroid, such as dexamethasone). Due to the limited aqueous solubility of most corticosteroids, compositions containing this type of anti-inflammatory agent are generally formulated as suspensions. TOBRADEX® (Tobramycin/Dexamethasone) Sterile Ophthalmic Suspension is an example of this type of composition. The present invention is directed to the provision of self-preserved tobramycin/corticosteroid suspensions of this type, and is particularly directed to the provision of an improved version of the existing TOBRADEX® formulation.

### DETAILED DESCRIPTION OF THE INVENTION

The self-preserved compositions of the present invention contain tobramycin in an amount effective to treat infections caused by microorganisms that are susceptible to this antibiotic. Such amount, which is simply referred to herein as an “anti-infective amount”, can be readily determined by those skilled in the art based on published minimum inhibitory concentration (“MIC”) data for tobramycin. The concentration of tobramycin in the compositions of the present invention will typically be in the range of 0.1 to 1.0 w/v % weight/volume percent (“w/v %”). The most preferred concentration for the ophthalmic compositions of the present invention is 0.3 w/v %.

Relative to bacteria, the USP 27 Antimicrobial Effectiveness Test requires that multi-dose ophthalmic compositions have sufficient antimicrobial activity to reduce an initial inoculum of approximately 10⁵ to 10⁶ bacteria by one log (i.e., a 90% reduction in the microorganism population) over a period of seven (7) days and by three logs (i.e., a 99.9% reduction in the microorganism population) over a period of fourteen (14) days, and requires that there cannot be any increase in the microorganism population following the conclusion of the fourteen day period. Relative to fungi, the USP standards require that the compositions maintain stasis (i.e., no growth) relative to the population of the initial inoculum over the entire 28 day test period.

The margin of error in calculating microorganism populations is generally accepted to be +/−0.5 logs. Accordingly, the term “stasis”, as utilized herein relative to the above-discussed USP standards, means that the initial fungi population cannot increase by more than 0.5 log orders, relative to the initial population.

The self-preserved tobramycin compositions of the present invention preferably contain borate in an amount effective to enhance the antimicrobial activity of the compositions. As used herein, the term “borate” includes boric acid, salts of boric acid, other pharmaceutically acceptable borates, and combinations thereof. The following borates are particularly preferred: boric acid, sodium borate, potassium borate, calcium borate, magnesium borate, manganese borate, and other such borate salts. The compositions of the present invention preferably contain one or more borates in an amount equivalent to a boric acid concentration of 0.02 to 0.25 moles per liter, more preferably 0.04 to 0.2 moles/liter, and most preferable 0.075 to 0.175 moles/liter.

The compositions of the present invention preferably also contain one or more polyols selected from the group consisting of propylene glycol, glycerol and combinations thereof. The combination of borate with such polyols results in the formation of borate/polyol complexes that further enhance the antimicrobial activity of the aqueous ophthalmic compositions of the present invention. The compositions of the present invention therefore preferably contain one or more borates, in the amounts specified above, and propylene glycol, glycerol or a combination thereof, at a total polyol concentration of 0.0125 to 0.15 moles/liter, more preferably 0.025 to 0.125 moles/liter. The use of propylene glycol at a concentration of 0.05 to 0.1 moles/liter is most preferred.

The compositions of the present invention preferably also contain one or more anti-inflammatory agents. The anti-inflammatory agents utilized in the present invention are broadly classified as steroidal or non-steroidal. The preferred steroidal anti-inflammatory agents are glucocorticoids.

The preferred glucocorticoids for ophthalmic and otic use include dexamethasone, loteprednol, rimexolone, prednisolone, fluorometholone, and hydrocortisone. The preferred glucocorticoids for nasal use include mometasone, fluticasone, beclomethasone, flunisolide, triamcinolone and budesonide.

The dexamethasone derivatives described in U.S. Pat. No. 5,223,493 (Boltralik) are also preferred steroidal anti-inflammatory agents, particularly with respect to compositions for treating ophthalmic inflammation. The following compounds are especially preferred: 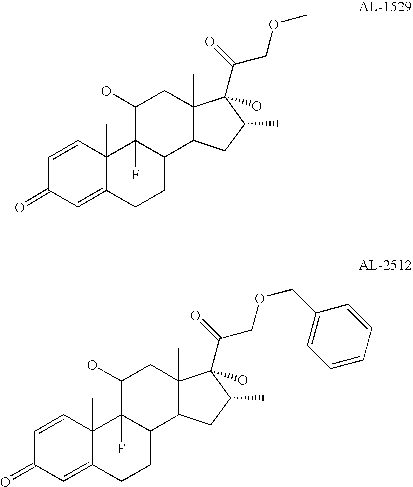
These compounds are referred to herein as “21-ether derivatives of dexamethasone”. The 21-benzyl ether derivative (i.e., compound AL-2512) is particularly preferred.

The preferred non-steroidal anti-inflammatory agents are: prostaglandin H synthetase inhibitors (Cox I or Cox II), also referred to as cyclooxygenase type I and type II inhibitors, such as diclofenac, flurbiprofen, ketorolac, suprofen, nepafenac, amfenac, indomethacin, naproxen, ibuprofen, bromfenac, ketoprofen, meclofenamate, piroxicam, sulindac, mefanamic acid, diflusinal, oxaprozin, tolmetin, fenoprofen, benoxaprofen, nabumetome, etodolac, phenylbutazone, aspirin, oxyphenbutazone, NCX-4016, HCT-1026, NCX-284, NCX-456, tenoxicam and carprofen; cyclooxygenase type II selective inhibitors, such as NS-398, vioxx, celecoxib, P54, etodolac, L-804600 and S-33516; PAF antagonists, such as SR-27417, A-137491, ABT-299, apafant, bepafant, minopafant, E-6123, BN-50727, nupafant and modipafant; PDE IV inhibitors, such as ariflo, torbafylline, rolipram, filaminast, piclamilast, cipamfylline, CG-1088, V-11294A, CT-2820, PD-168787, CP-293121, DWP-205297, CP-220629, SH-636, BAY-19-8004, and roflumilast; inhibitors of cytokine production, such as inhibitors of the NFkB transcription factor; or other anti-inflammatory agents known to those skilled in the art.

The concentrations of the anti-inflammatory agents contained in the compositions of the present invention will vary based on the agent or agents selected and the type of inflammation being treated. The concentrations will be sufficient to reduce inflammation in the targeted ophthalmic, otic or nasal tissues following topical application of the compositions to those tissues. Such an amount is referred to herein as “an anti-inflammatory effective amount”. The compositions of the present invention will typically contain one or more anti-inflammatory agents in an amount of from about 0.01 to about 1.0 wt. %.

The ophthalmic, otic and nasal compositions of the present invention will contain tobramycin and preferably one or more anti-inflammatory agents, in pharmaceutically acceptable vehicles. The compositions will typically have a pH in the range of 4.5 to 8.0. The ophthalmic compositions must also be formulated to have osmotic values that are compatible with the aqueous humor of the eye and ophthalmic tissues. Such osmotic values will generally be in the range of from about 200 to about 400 milliosmoles per kilogram of water (“mOsm/kg”), but will preferably be about 280 mOsm/kg.

The solubility of the components of the present compositions may be enhanced by a surfactant or other appropriate co-solvent in the composition. Such co-solvents include tyloxopol, polysorbate 20, 60, and 80, polyoxyethylene/polyoxypropylene surfactants (e.g., Pluronic F-68, F-84 and P-103), cyclodextrin, or other agents known to those skilled in the art. Typically such co-solvents are employed at a level of from 0.01% to 2% by weight.

The use of viscosity enhancing agents to provide the compositions of the invention with viscosities greater than the viscosity of simple aqueous solutions may be desirable to increase ocular absorption of the active compounds by the target tissues or increase the retention time in the eye, ear or nose. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, carbomers or other agents know to those skilled in the art. Such agents are typically employed at a level of from 0.01% to 2% by weight.

The compositions are typically administered to the affected ophthalmic, otic or nasal tissues by topically applying one to four drops of a sterile solution or suspension, or a comparable amount of an ointment, gel or other solid or semisolid composition, one to four times per day. However, the compositions may also be formulated as irrigating solutions that are applied to the affected ophthalmic, otic or nasal tissues during surgical procedures.

The following examples are presented to further illustrate the preferred embodiments of the invention.

### EXAMPLE 1

The following formulation is currently marketed by Alcon Laboratories, Inc. as TOBRADEX® (Tobramycin/Dexamethasone) Sterile Ophthalmic Suspension:

TABLE 1

| Component | Concentration (w/v %) |
|---|---|
| Dexamethasone, Micronized USP | 0.10% + 5% excess |
| Tobramycin, USP | 0.30 + 5% excess |
| Benzalkonium Chloride Solution (10%) NF | 0.10% + 10% excess |
| Edetate Disodium, USP | 0.01% |
| Sodium Chloride, USP | 0.3% |
| Sodium Sulfate, USP | 1.2% |
| Tyloxapol, USP | 0.05% |
| Hydroxyethylcellulose | 0.25% |
| Sulfuric Acid and/or Sodium | QS for pH adjustment |
| Hydroxide, NF | to 5.5 ± 0.5 |
| Purified Water, USP | QS to 100% |

The present inventors have discovered that it is possible to satisfy the USP 27 preservative efficacy requirements without including benzalkonium chloride (“BAC”) in the above-described formulation. However, in order to allow the modified formulation to consistently and reliably satisfy the USP requirements, it is preferable to include a borate/polyol complex in the composition. The findings of the present inventors relative to the selection of an appropriate borate/polyol complex are discussed in Examples 2 and 3, below.

### EXAMPLE 2

As discussed above, the inventors initially attempted to replace the BAC contained in the existing TOBRADEX® formulation with a borate/polyol complex consisting of boric acid and sorbitol. One of the formulations tested by the inventors is shown in Table 2 below:

TABLE 2

| Component | Concentration (w/v %) | | |
|---|---|---|---|
| Tobramycin | 0.3 | | |
| Dexamethasone | 0.1 | | |
| HEC 250HR | 0.25 | | |
| Sorbitol | 1 | | |
| Boric Acid | 1 | | |
| Tyloxapol | 0.05 | | |
| NaOH | pH 5.7 | | |
| NCl | pH 5.7 | | |
| Purified Water | QS for 100% of desired volume | | |
| Osmolality mOsm/kg | 193 | | |
| | Log Order Reductions | | |
| Microorganism | 6 hours | 24 hours | 7 days |
| Staph. Aureus | 4.5 | 4.8 | — |
| Pseudomonas aeruginosa | 4.9 | 4.9 | — |
| E. coli | 4.8 | 4.8 | — |
| Candida albicans | — | — | 3.8 |
| Aspergillus niger | — | — | 4.6 |

The formulation shown in Table 2 satisfied the USP preservative efficacy requirements. However, when additional compositions containing sorbitol were tested (see Example 3, below), it was discovered that sorbitol was adversely affecting the chemical stability of tobramycin.

### EXAMPLE 3

Several self-preserved tobramycin/dexamethasone formulations were tested in order to evaluate the chemical stability of tobramycin in the presence of various types of borate/polyol complexes. The formulations tested and stability data are presented in Tables 3a, 3b and 3c, below.

TABLE 3a
Accelerated Thermal Stability Studies of Tobramycin and Dexamethasone
in Prototype Suspension Formulations Packaged in 8.0 mL EtO
Sterilized Natural Drop-Tainer ®

| | | FID 107152 |
|---|---|---|
| | | Batch 04-37012 |
| | | pH 5.7 |
| | | 0.3% Tobramycin |
| | | 0.1% Dex-OH |
| | | 0.25% HEC 250HR |
| | | 1.0% Boric Acid |
| | | 0.6% Propylene Glycol |
| | | 0.05% Tyloxapol |
| | | 0.18% Na Sulfate |
| Time Pull | Tests | 40° C. |
| Initial | Observation | White |
| | % Weight Loss | na |
| | % Tobramycin (% N) | 101, 101 (100) |
| | % Dex_OH (% N) | 124, 124 (100) |
| | pH | 5.65, 5.65 |
| | Osmolality | 293, 294 |
| 4 weeks | Observation | White |
| | % Weight Loss | 0.72, 0.70 |
| | % Tobramycin (% N) | 104, 103 (102) |
| | % Dex_OH (% N) | 124, 124 (100) |
| | pH | 5.54, 5.58 |
| | Osmolality | 293, 294 |
| 8 weeks | Observation | White |
| | % Weight Loss | 1.32, 1.30 |
| | % Tobramycin (% N) | 103, 103 (102) |
| | % Dex_OH (% N) | 125, 122 (100) |
| | pH | 5.55, 5.54 |
| | Osmolality | 294, 295 |
| 12 weeks | Observation | White |
| | % Weight Loss | 1.96, 2.00 |
| | % Tobramycin (% N) | 103, 103 (102) |
| | % Dex_OH (% N) | 124, 123 (100) |
| | pH | 5.56, 5.55 |
| | Osmolality | 296, 297 |
| 16 weeks | Observation | White |
| | % Weight Loss | 2.56, 2.54 |
| | % Tobramycin (% N) | 101, 101 (100) |
| | % Dex_OH (% N) | 122, 122 (98) |
| | pH | 5.49, 5.51 |
| | Osmolality | 297, 298 |

TABLE 3b
Study of Tobramycin and Dexamethasone in Ophthalmic Suspension Formulations
Packaged in 8.0 mL EtO Sterilized Natural Drop-Tainer ®

| | | FID 107294 | FID 107295 | FID 107296 |
|---|---|---|---|---|
| | | Batch 04-37004 | Batch 04-37005 | Batch 04-37007 |
| | | pH 5.7 | pH 5.7 | pH 5.7 |
| | | 0.3% Tobramycin | 0.3% Tobramycin | 0.3% Tobramycin |
| | | 0.1% Dex-OH | 0.1% Dex-OH | 0.1% Dex-OH |
| | | 0.25% HEC 250HR | 0.25% HEC 250HR | 0.25% HEC 250HR |
| | | 1.0% Boric Acid | 1.0% Boric Acid | 1.0% Boric Acid |
| | | 0.05% Tyloxapol | 0.05% Tyloxapol | 0.05% Tyloxapol |
| | | 0.18% Na Sulfate | 0.18% Na Sulfate | 0.18% Na Sulfate |
| Time Pull | Tests | 1.27% Mannitol | 0.65% Glycerin | 1.06% Xylitol |
| Initial | Observation | White | White | White |
| | % Weight Loss | na | na | na |
| | % Tobramycin (% N) | 96, 97 (100) | 103, 103 (100) | 104, 103 (100) |
| | % Dex_OH (% N) | 103, 103 (100) | 102, 101 (100) | 103, 103 (100) |
| | pH | 5.77, 5.77 | 5.79, 5.77 | 5.75, 5.75 |
| | Osmolality | 229, 229 | 287, 287 | 247, 246 |
| 4 weeks at | Observation | White | White | White |
| 40° C. | % Weight Loss | 0.76, 0.76 | 0.76, 0.74 | 0.74, 0.72 |
| | % Tobramycin (% N) | 97, 97 (101) | 103, 103 (100) | 103, 103 (99) |
| | % Dex_OH (% N) | 100, 102 (98) | 101, 101 (100) | 102, 102 (99) |
| | pH | 5.75, 5.76 | 5.77, 5.74 | 5.73, 5.73 |
| | Osmolality | 221, 229 | 284, 287 | 248, 247 |
| 8 weeks at | Observation | White | White | White |
| 40° C. | % Weight Loss | 1.28, 1.30 | 1.30, 1.28 | 1.26, 1.26 |
| | % Tobramycin (% N) | 96, 95 (99) | 103, 103 (100) | 101, 102 (98) |
| | % Dex_OH (% N) | 99, 99 (96) | 101, 100 (99) | 100, 101 (98) |
| | pH | 5.71, 5.70 | 5.71, 5.66 | 5.68, 5.67 |
| | Osmolality | 233, 229 | 286, 289 | 249, 248 |
| 12 weeks at | Observation | White | White | Lt. Yellow |
| 40° C. | % Weight Loss | 1.98, 2.00 | 1.98, 1.96 | 1.98, 1.98 |
| | % Tobramycin (% N) | 95, 95 (98) | 103, 103 (100) | 100, 100 (97) |
| | % Dex_OH (% N) | 98, 97 (95) | 99, 98 (97) | 98, 99 (96) |
| | pH | 5.73, 5.74 | 5.71, 5.71 | 5.71, 5.71 |
| | Osmolality | 233, 233 | 288, 289 | 249, 250 |
| 16 weeks at | Observation | White | White | Lt. Yellow |
| 40° C. | % Weight Loss | 2.60, 2.58 | 2.60, 2.60 | 2.54, 2.56 |
| | % Tobramycin (% N) | 95, 94 (98) | 101, 102 (99) | 99, 101 (97) |
| | % Dex_OH (% N) | 96, 98 (94) | 99, 99 (98) | 97, 98 (95) |
| | pH | 5.72, 5.71 | 5.71, 5.72 | 5.71, 5.70 |
| | Osmolality | 235, 233 | 290, 291 | 251, 252 |

TABLE 3c
Study of Tobramycin and Dexamethasone in Ophthalmic Suspension Formulations
Packaged in 8.0 mL EtO Sterilized Natural Drop-Tainer ®

| | | FID 107298 | FID 107300 | FID 107300 |
|---|---|---|---|---|
| | | Batch 04-37008 | Batch 04-37009 | Batch 04-37010 |
| | | pH 5.7 | pH 5.7 | pH 5.7 |
| | | 0.3% Tobramycin | 0.3% Tobramycin | 0.3% Tobramycin |
| | | 0.1% Dex-OH | 0.1% Dex-OH | 0.1% Dex-OH |
| | | 0.25% HEC 250HR | 0.25% HEC 250HR | 0.25% HEC 250HR |
| | | 1.0% Boric Acid | 1.0% Boric Acid | 1.0% Boric Acid |
| | | 0.05% Tyloxapol | 0.05% Tyloxapol | 0.05% Tyloxapol |
| | | 0.18% Na Sulfate | 0.18% Na Sulfate | 0.18% Na Sulfate |
| Time Pull | Tests | 2.31% Sucrose | 1.37% Sorbitol | 1.37% Sorbitol |
| Initial | Observation | White | White | White |
| | % Weight Loss | na | na | na |
| | % Tobramycin (% N) | 103, 102 (100) | 103, 104 (100) | 103, 103 (100) |
| | % Dex_OH (% N) | 104, 105 (100) | 102, 102 (100) | 102, 102 (100) |
| | pH | 5.73, 5.71 | 5.81, 5.79 | 5.75, 5.75 |
| | Osmolality | 287, 288 | 233, 231 | 234, 233 |
| 4 weeks at | Observation | White | White | White |
| 40° C. | % Weight Loss | 0.72, 0.72 | 0.72, 0.74 | 0.72, 0.72 |
| | % Tobramycin (% N) | 103, 104 (101) | 102, 103 (99) | 102, 102 (99) |
| | % Dex_OH (% N) | 103, 104 (99) | 99, 100 (98) | 100, 99 (98) |
| | pH | 5.70, 5.62 | 5.77, 5.75 | 5.73, 5.73 |
| | Osmolality | 287, 288 | 233, 233 | 233, 234 |
| 8 weeks at | Observation | White | White | White |
| 40° C. | % Weight Loss | 1.28, 1.28 | 1.26, 1.24 | 1.24, 1.26 |
| | % Tobramycin (% N) | 103, 102 (100) | 102, 102 (99) | 102, 102 (99) |
| | % Dex_OH (% N) | 103, 102 (98) | 98, 97 (96) | 99, 99 (97) |
| | pH | 5.56, 5.52 | 5.72, 5.72 | 5.68, 5.68 |
| | Osmolality | 288, 287 | 232, 233 | 236, 233 |
| 12 weeks | Observation | Yellow | Yellow | Yellow |
| at 40° C. | % Weight Loss | 1.96, 1.98 | 1.98, 1.94 | 1.94, 1.96 |
| | % Tobramycin (% N) | 100, 100 (98) | 100, 99 (96) | 100, 99 (97) |
| | % Dex_OH (% N) | 102, 102 (98) | 95, 95 (93) | 96, 96 (94) |
| | pH | 5.42, 5.42 | 5.74, 5.74 | 5.72, 5.71 |
| | Osmolality | 293, 292 | 235, 235 | 235, 235 |
| 16 weeks | Observation | Yellow | Yellow | Yellow |
| at 40° C. | % Weight Loss | 2.60, 2.60 | 2.54, 2.54 | 2.60, 2.56 |
| | % Tobramycin (% N) | 103, 103 (100) | 101, 103 (99) | 104, 103 (100) |
| | % Dex_OH (% N) | 103, 103 (99) | 94, 95 (93) | 96, 96 (94) |
| | pH | 5.30, 5.22 | 5.74, 5.74 | 5.71, 5.71 |
| | Osmolality | 298, 296 | 236, 238 | 238, 238 |

### EXAMPLE 4

The formulation shown in Table 4, below, contains the polyol propylene glycol, instead of sorbitol. The results presented in Table 3a show that this formulation did not adversely affect the stability of tobramycin. As shown in Table 4, this formulation has demonstrated sufficient antimicrobial activity to satisfy the USP 27 Antimicrobial Effectiveness Test.

TABLE 4

| Component | Concentration (w/v %) | | | | |
|---|---|---|---|---|---|
| Tobramycin | 0.3 | | | | |
| Dexamethasone | 0.1 | | | | |
| HEC 250HR | 0.25 | | | | |
| Boric acid | 1.0 | | | | |
| Sorbitol | — | | | | |
| Propylene glycol | 0.6 | | | | |
| Tyloxapol | 0.05 | | | | |
| Sodium Sulfate | 0.18 | | | | |
| NaOH | QS pH 5.7 | | | | |
| Sulfuric acid | QS pH 5.7 | | | | |
| Purified Water | QS for 100% of desired volume | | | | |
| Osmolality, mOsm/kg | 279 | | | | |
| | Log Order Reductions | | | | |
| Microorganism | 6 hours | 24 hours | 7 days | 14 days | 28 days |
| Staph. Aureus | 3.4 | 4.6 | 4.9 | 4.9 | 4.9 |
| Pseudomonas | 4.9 | 4.9 | 4.9 | 4.9 | 4.9 |
| aeruginosa | | | | | |
| E. coli | 5.0 | 5.0 | 5.1 | 5.1 | 5.1 |
| Candida albicans | — | — | 1.5 | 1.4 | 1.0 |
| Aspergillus niger | — | — | 1.5 | 2.1 | 3.7 |

We claim:

1. A self-preserved pharmaceutical compositions packaged as a multi-dose product, said composition comprising an anti-infective amount of tobramycin, and an aqueous, pharmaceutically acceptable vehicle therefor.

2. A self-preserved composition according to claim 1, wherein the composition further comprises an amount of a borate sufficient to enhance the antimicrobial activity of the composition relative to Aspergillus niger or Candida albicans.

3. A self-preserved composition according to claim 1, wherein the composition further comprises an amount of a borate/polyol complex sufficient to enhance the antimicrobial activity of the composition relative to Aspergillus niger or Candida albicans.

4. A self-preserved composition according to claim 3, wherein the borate/polyol complex is selected from the group consisting of borate/propylene glycol, borate/glycerol, and combinations thereof.
